(19) United States
(12) Patent Application Publication
(10) Pub. No.: US 2021/0327581 A1
(43) Pub. Date: Oct. 21, 2021

(54) DIAGNOSTIC DEVICE FOR IMPROVING TRICHOTILLOMANIA AND DIAGNOSTIC METHOD OF IMPROVING TRICHOTILLOMANIA

(71) Applicant: Japan Trichotillomania Improvement Association, Hyogo (JP)

(72) Inventor: Hidehiro NAKAGAWA, Hyogo (JP)

(21) Appl. No.: 17/205,150

(22) Filed: Mar. 18, 2021

(30) Foreign Application Priority Data
Apr. 16, 2020 (JP) 2020-073418

(51) Int. Cl. G16H 50/20; G16H 10/60; G16H 70/40

(52) U.S. Cl. CPC G16H 50/20; G16H 70/40; G16H 10/60

(57) ABSTRACT

Provided herein are a diagnostic device for improving trichotillomania and a diagnostic method of improving trichotillomania capable of quantifying: and visualizing a possibility of improvement in trichotillomania. A diagnostic device for improving trichotillomania 20 includes a customer information storage unit 25A to store customer information, a diagnostic information storage unit 25B storing an impulse factor of pulling out hair and an improvement factor as diagnostic information, a master improvement factor storage unit 25C preliminarily making the improvement factor correspond to a point and store the improvement factor with the point, a diagnostic score calculation unit 22 calculating a quantified possibility of improvement of trichotillomania, and a diagnostic result issuing unit 23 to issue a diagnostic result including the diagnostic score.

### CROSS REFERENCE TO RELATED APPLICATION

This application claims priority under 35 U.S.C. 119 to Japanese patent application No. 2020-073418 filed Apr. 16, 2020, the entire text of which is specifically incorporated herein by reference.

### BACKGROUND OF THE INVENTION

### Field of the Invention

The present invention relates to a diagnostic device for improving trichotillomania and a diagnostic method of improving trichotillomania to diagnose a possibility of improvement in trichotillomania.

As described in “Trichotillomania” from Wikipedia searched Apr. 1, 2020, Internet URL:https://ja.wikipedia.org/wiki/%E6%8A%C%E6%AF%9B%E7%97%87, trichotillomania is a mental disorder that causes a spot baldness by a habit of pulling out normal hair. Trichotillomania is also called as a hair pulling habit, or baldness because scalp hair is pulled out in most cases. Trichotillomania is included in an impulse-control disorder according to DSM-IV and ICD-10. Some people sometimes unconsciously pull their hair out without awareness. Some people last that symptom for five or six years.

### SUMMARY OF THE INVENTION

People follow their hunches regarding improvement of trichotillomania at present. The above-described reference of “Trichotillomania” from Wikipedia describes, with respect to treatment, “effective medicine for trichotillomania has not been developed at this time”, and “because effective treatment is unavailable, a major problem is that it is uncertain which hospital people should go”. In recent years, where consultation for improvement of trichotillomania is performed changes from hospitals to barbershops or hair salons, but it is very difficult to decide whether people consult with a physician, a barber, or a hairdresser.

An object of the present invention is to provide a diagnostic device for improving trichotillomania and a diagnostic method of improving trichotillomania capable of quantifying and visualizing a possibility of improvement in trichotillomania.

As an embodiment of the present invention, a diagnostic device for improving trichotillomania includes: a storage unit for information of a person to be diagnosed configured to store the information of the person to be diagnosed when the information of the person to be diagnosed including an ID of the person to be diagnosed is inputted from a user terminal that is communicably connected via a network; a diagnostic information storage unit configured to store an impulse factor of pulling out hair as diagnostic information when the impulse factor of pulling out hair including the ID of the person to be diagnosed is inputted, and an improvement factor is stored as the diagnostic information when the improvement factor including the ID of the person to be diagnosed is inputted; a master improvement factor storage unit configured to preliminarily make the improvement factor correspond to a point and store the improvement factor with the point; a diagnostic score calculation unit configured to calculate a diagnostic score that is a quantified possibility of improving trichotillomania by extracting all of the improvement factors based on the ID of the person to be diagnosed, extracting the point corresponding to all of the improvement factors from the master improvement factor storage unit, and adding all of the points that are extracted when the user terminal requests the diagnostic score with the ID of the person to be diagnosed specified; a. diagnostic result issuing unit configured to issue a diagnostic result including the diagnostic score when the user terminal requests an issuance of the diagnostic result with the ID of the person to be diagnosed; and a point updating unit configured to update the point stored in the master improvement factor storage unit by using a different algorithm for every category of the improvement factor.

As an another embodiment of the present invention, a diagnostic method of improving trichotillomania by using a computer includes the steps of: storing information of a person to be diagnosed including an ID of the person to be diagnosed in a storage unit for information of the person to be diagnosed when the information of the person to be diagnosed including the ID of the person to be diagnosed is inputted; storing an impulse factor of pulling out hair as diagnostic information in a diagnostic information storage unit when the impulse factor of pulling out hair including the ID of the person to be diagnosed is inputted, and storing an improvement factor as the diagnostic information in a diagnostic information storage unit when the improvement factor including the ID of the person to be diagnosed is inputted; storing the improvement factor with the point in a master improvement factor storage unit by preliminarily making the improvement factor correspond to the point; calculating a diagnostic score that is a quantified possibility of improving trichotillomania by extracting all of the improvement factors based on the ID of the person to be diagnosed from the diagnostic information storage unit, extracting the points corresponding to all of the improvement factors from the master improvement factor storage unit, and adding all of the points that are extracted, when the user terminal requests the diagnostic score with the ID of the person to be diagnosed specified; issuing a diagnostic result including the diagnostic score when the user terminal requests an issuance of the diagnostic result with the ID of the person to be diagnosed specified; and updating the point stored in the master improvement factor storage unit by using a different algorithm for every category of the improvement factor.

According to the present invention, it is made possible to provide the diagnostic device for improving trichotillomania and the diagnostic method of improving trichotillomania capable of quantifying and visualizing the possibility of improvement in trichotillomania.

### BRIEF DESCRIPTION OF DRAWINGS

FIG. 1 shows a configuration of a diagnostic system for improving trichotillomania according to an embodiment.

FIG. 2 is a functional block diagram showing a diagnostic device for improving trichotillomania according to the embodiment.

FIGS. 3A to 3C are database configurations stored in a storage unit according to the embodiment. FIG. 3A is the database configuration stored in a customer information storage unit, FIG. 3B is the database configuration stored in a diagnostic information storage unit, and

FIG. 3C is the database configuration stored in a master improvement factor storage unit.

FIG. 4 is a flowchart showing operations of the diagnostic system for improving trichotillomania according to the embodiment.

FIG. 5 shows a screen transition displayed on a user terminal according to the embodiment.

FIG. 6 shows a screen transition displayed on the user terminal according to the embodiment.

FIG. 7 shows a screen transition displayed on the user terminal according to the embodiment.

FIG. 8 shows a screen transition displayed on the user terminal according to the embodiment.

FIG. 9A and FIG. 9B are explanatory diagrams of statical data according to the embodiment. FIG. 9A is a schematic diagram showing a diagnostic score and a possibility of improvement and FIG. 9B is a graph showing a relation between the improvement factor and improvement in speed.

FIG. 10 shows a configuration of hardware of the diagnostic device for improving trichotillomania according to the embodiment.

### DETAILED DESCRIPTION OF EMBODIMENTS

Embodiments of the present invention are described below with reference to the drawings. Note that embodiments as described below are only examples. In other words, the embodiments described below may be variously modified without departing from the scope of the subject matter. In drawings, a part where the same reference sign is used indicates the same or identical part unless otherwise noted.

[Introduction]

Technologies and information to help improvement of trichotillomania are desired to be provided to people from preschool children to adults who suffer from trichotillomania across the country. At present, low-effective medication is employed in medical treatment, and only supportive measures including wigs, and hair growth and/or hair restoration are employed in barbershops and hair salons. An inventor of the present invention quantifies and visualizes a possibility of improvement of trichotillomania based on statistical data of total more than 2,000 people.

[Configuration of System]

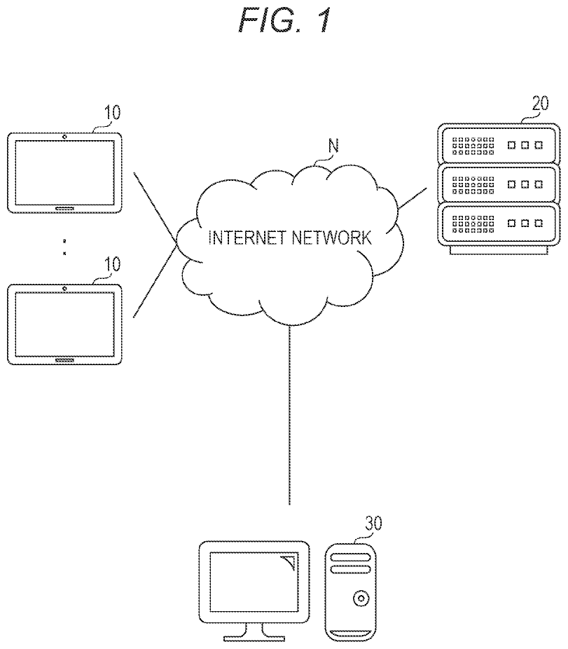

FIG. 1 shows a configuration of a diagnostic system for improving trichotillomania according to an embodiment. As shown in FIG I, a user terminal 10, a diagnostic device for improving trichotillomania 20, and an administrative terminal 30 are connected to a network N such as an internet network.

The user terminal 10 is a computer operated by a member of the system. In the embodiment, descriptions are made by supposing that a hairdresser as a member uses a tablet-type user terminal 10 when the hairdresser hears from a customer who suffers from trichotillomania and visits a hair salon.

The diagnostic device for improving trichotillomania 20 is a computer to diagnose the possibility of improvement of trichotillomania based on a result of what the hairdresser hears from the customer. The diagnostic device for improving trichotillomania 20 can be achieved by using a generic computer. Details regarding functions of the diagnostic device for improving trichotillomania 20 are described below.

The administrative terminal 30 is a computer operated by an administrator of the system. The administrative terminal 30 can be also achieved by using the generic computer. The administrator sets the system entirely by using the administrative terminal 30.

[Diagnostic Device for Improving Trichotillomania]

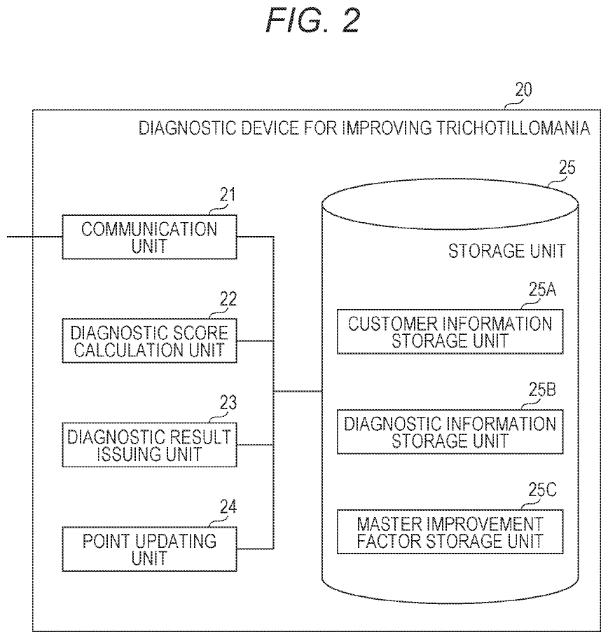

FIG. 2 is a functional block diagram showing the diagnostic device for improving trichotillomania 20. As shown in FIG. 2, the diagnostic device for improving trichotillomania. 20 includes a communication unit 21, a diagnostic score calculation unit 22. a diagnostic result issuing unit 23, a point updating unit 24, and a storage unit 25. Such respective functional units are achieved by CPU's execution of a program loaded on a memory in a computer as described below.

The communication unit 21 controls communication with other terminal. Specifically, the communication unit 21 communicates the user terminal 10 and the administrative terminal 30 via the network N.

The storage unit 25 stores various information (database). The storage unit 25 includes a customer information storage unit 25A, a diagnostic information storage unit 25B, and a master improvement factor storage unit 25C.

The customer information storage unit 25A stores customer information. The customer information is information about a customer to be diagnosed. Descriptions are made by supposing that a person to be diagnosed is a customer who visits a hair salon, but a person to be diagnosed may be a patient who visits a hospital.

The diagnostic information storage unit 25B stores an impulse factor of pulling out hair as well as an improvement factor as diagnostic information. The impulse factor of pulling out hair is information that indicates a factor that leads to an impulse to pull out hair, including “matters related to a school and/or an entrance exam”, “matters related to a relationship with friends”, and “family issues”. The improvement factor is information that indicates a factor that leads to deterrence to impulsively pulling out hair, including “favorite snack”, “favorite music”, and “pet animals”.

The master improvement factor storage unit 25C preliminarily makes the improvement factor correspond to a point and store the improvement factor with the point. Such process of storing the point may be performed automatically with the execution of a program or manually by the administrator using the administrative terminal 30.

A diagnostic score calculation unit 22 calculates a diagnostic score. The diagnostic score is a quantified score of the possibility of improvement of trichotillomania and can be restated as a point of the possibility of improvement.

A diagnostic result issuing unit 23 issues a diagnostic result with the diagnostic score. The diagnostic result is a quantified and visualized possibility of improvement of trichotillomania. The members of system may be hairdressers and/or barbers, as well as health care practitioners. The diagnostic result is named as a “prescription” below, but what is named is not limited thereto.

The point updating unit 24 updates the point stored in the master improvement factor storage unit 25C. Such process of updating the point may be performed automatically with the execution of the program or manually with the administrator using the administrative terminal 30.

[Configuration of Database]

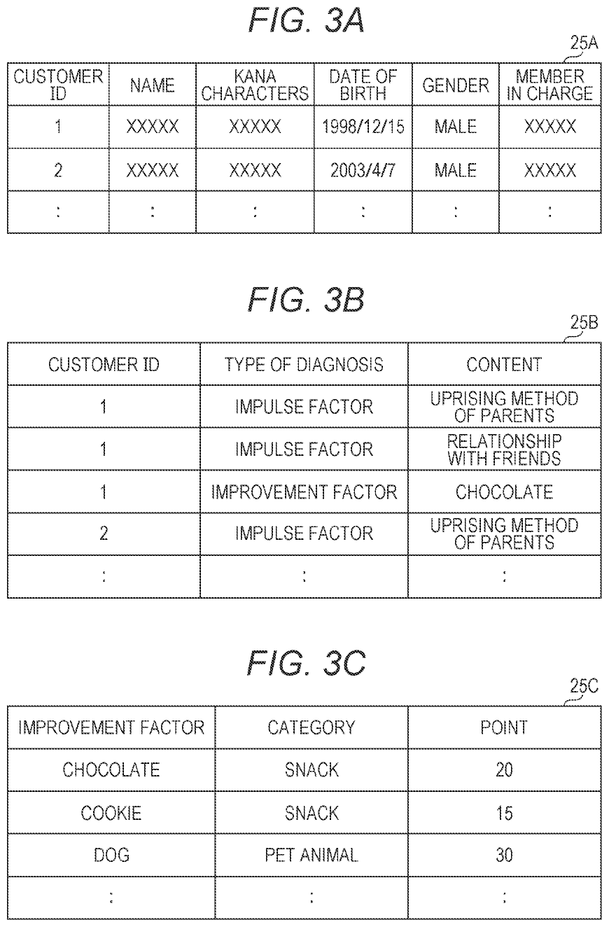

FIG. 3A to FIG. 3C show configurations of databases stored in the storage unit 25. Inner configurations of the respective databases are exemplified below with reference to FIG. 3A to FIG. 3C.

As shown in FIG. 3A, the customer information storage unit 25A stores the customer information including a customer ID, a name, a date of birth, a gender, and a member in charge. The customer ID is identification data of a customer. The name, the date of birth, and the gender are the name, the date of birth, and the gender of the customer. The member in charge is the the name of the member in charge of the customer (the hairdresser).

As shown in FIG. 3B, the diagnostic information storage unit 25B stores the diagnostic information including a type of diagnosis and content. The type of diagnosis is a type of the diagnostic information that matches either the impulse factor of pulling out hair or the improvement factor. The content is a specific content of the diagnostic information. Referring to a customer having a customer ID “1 (one)”, an uprising method of parents and a relationship with friends are exemplified as the impulse factors of pulling out hair, and chocolate is exemplified as the improvement factor.

As shown in FIG. 3C, the master improvement factor storage unit 25C stores the improvement factor, a category, and the point by making the improvement factor, a category, and the point correspond to each other. The category refers to where the improvement factor belongs to. The point refers to a point given to the improvement factor. Herein, “chocolate” as the improvement factor is categorized as snack so that 20 points are given. “Cookie” as the improvement factor is categorized as the snack so that 15 points are given, A “dog” as the improvement factor is categorized as pet animals so that 30 points are given.

[Diagnostic Score]

Next, a calculation method of the diagnostic score is specifically described. The diagnostic score calculation unit 22 refers to the storage unit 25 to calculate total points given to the improvement factor as the diagnostic score. For example, the diagnostic score of the customer ID “1 (one)” as shown in FIG. 3A to FIG. 3C is calculated as 20 points that are given by chocolate because only chocolate is the improvement factor. When the improvement factor is two or more, total points are calculated by adding all of the points given by the improvement factors, for example, 10 points by snack+20 points by music, etc.

[Updating Process]

Updating process of the scores stored in the master improvement factor storage unit 25C is described. The point updating unit 24 updates the points stored in the master improvement factor storage unit 25C by using a different algorithm for every category of the improvement factor. Thus, huge amounts of data (including questionnaire results) are processed by the different algorithm for every category so as to optimize allocations of the points.

For example, in the category of “snack”, the points stored in the master improvement factor storage unit 25C may be updated based on sugar content. Sugar affects serotonin that is chemical in the brain. Higher an item has sugar content, higher the possibility of improving trichotillomania is, thereby preferably making allocation of the points larger to such item.

Alternatively, responding to a boundary point of the diagnostic score where trichotillomania is improved in high probability in the statical data obtained in barbershops and/or hair salons, the point updating unit 24 may allocate the points to be stored in the master improvement factor storage unit 25C. In the embodiment, the boundary points of the diagnostic score where trichotillomania are improved by 90 percent or more are set as 150 points (described below), and 20 points are given to chocolate. Thus, when the boundary points of the diagnostic score where trichotillomania is improved by 90 percent or more are changed to 300 points, 40 points may be given to chocolate. Needless to say, the administrator is able to slightly adjust these points manually.

[Operation]

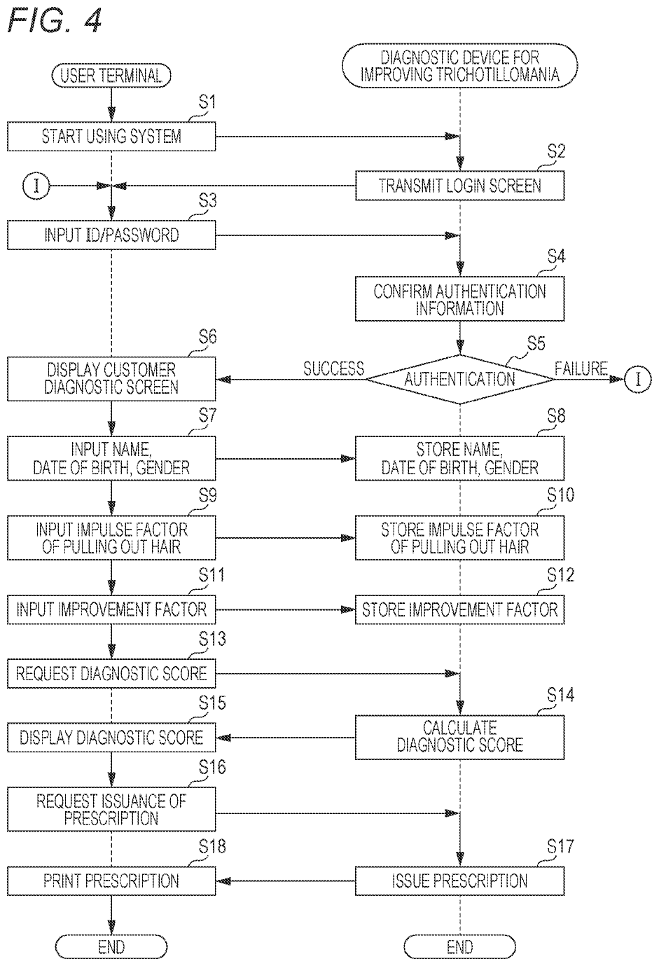

FIG. 4 is a flowchart showing operation of the system. Herein, also supposing that a hairdresser hears from a customer suffering from trichotillomania by operating the user terminal 10.

Firstly, the user terminal 10 instructs start of using the system (a step Si), the instruction of start of using is transmitted to the diagnostic device for improving trichotillomania 20. The diagnostic device for improving trichotillomania 20 returns a login screen to the user terminal 10 (a step S2).

Subsequently, an ID and a password are inputted on the login screen displayed on the user terminal 10 (a step S3), authentication information of the system is transmitted to the diagnostic device for improving trichotillomania 20. Thus, the diagnostic device for improving trichotillomania 20 confirms the authentication information (a step S4). When authentication is successful, the diagnostic device for improving trichotillomania 20 returns a customer diagnostic screen to the user terminal 10 (steps S5 to S6). In case the authentication is failed, the diagnostic device for improving trichotillomania 20 transmits a failure to the user terminal 10 and the operation is returned to the login screen(from steps S5 to S3).

Subsequently, a name, a date of birth, and a gender are inputted on the customer diagnostic screen displayed on the user terminal 10 (a step S7), the customer information is transmitted to the diagnostic device for improving trichotillomania 20. Thus, the customer information is stored in the customer information storage unit 25A in the diagnostic device for improving trichotillomania 20 (a step S8).

Subsequently, the the impulse factor of pulling out hair is inputted on the customer diagnostic screen displayed on the user terminal 10 (a step S9), the impulse factor of pulling out hair is transmitted to the diagnostic device for improving trichotillomania 20. Thus, the impulse factor of pulling out hair is stored in the diagnostic information storage unit 25B in the diagnostic device for improving trichotillomania 20 (a step S10).

Subsequently, the improvement factor is inputted on the customer diagnostic screen displayed on the user terminal 10 (a step S11), the improvement factor is transmitted to the diagnostic device for improving trichotillomania 20. Thus, the improvement factor is stored in the diagnostic information storage unit 25B in the diagnostic device for improving trichotillomania 20 (a step S12).

When the customer's improvement factor is transmitted in a state where the impulse factor of pulling out hair is not stored in the diagnostic information storage unit 25B, the diagnostic device for improving trichotillomania 20 may be set to return error to the user terminal 10. In this manner, it is made possible that the user terminal 10 accepts input of the improvement factor only after the impulse factor of pulling out hair is inputted (described below).

Subsequently, the diagnostic score is requested on the customer diagnostic screen displayed on the user terminal 10 (a step S13), the request for the diagnostic score is transmitted to the diagnostic device for improving trichotillomania 20. Thus, the diagnostic device for improving trichotillomania 20 calculates the diagnostic score and returns the score to the user terminal 10 (steps S14 to S15).

Subsequently, an issuance of a prescription is requested on the customer diagnostic screen displayed on the user terminal 10 (a step S16), the request for the issuance of the prescription is transmitted to the diagnostic device for improving trichotillomania 20, In this manner, the diagnostic device for improving trichotillomania 20 issues the prescription such as in a portable document format (PDF) file and returns the prescription to the user terminal 10 (a step 17).

Finally, the prescription is printed by the user terminal 10 and a printed prescription is submitted to the customer (a step S18). Various methods are used for submitting the prescription. For example, the prescription may be stored in a computer-readable storage medium to submit to the customer. Alternatively, the prescription may be attached to an email to send to an email address specified by the customer.

[Screen Transition]

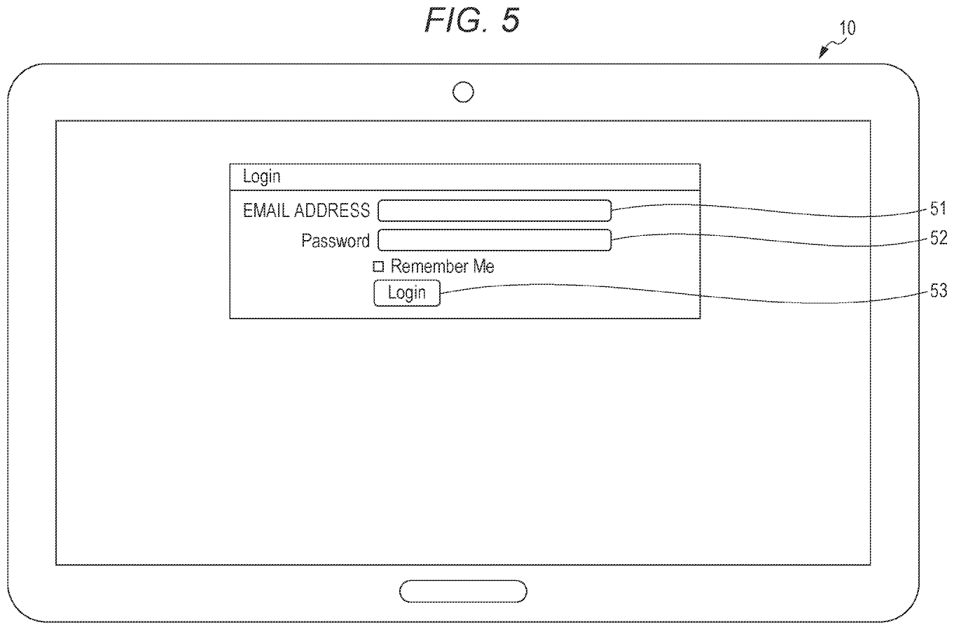

FIG. 5 to FIG. 8 show screen transitions displayed on the user terminal 10. With reference to FIG. 5 to FIG. 8, the configuration of the system is more specifically described below.

FIG. 5 is a login screen displayed on the user terminal 10. The login screen includes an ID input box 51, a password input box 52, and a login button to instruct login 53. Such login screen is widely known, so detailed description thereof is omitted here.

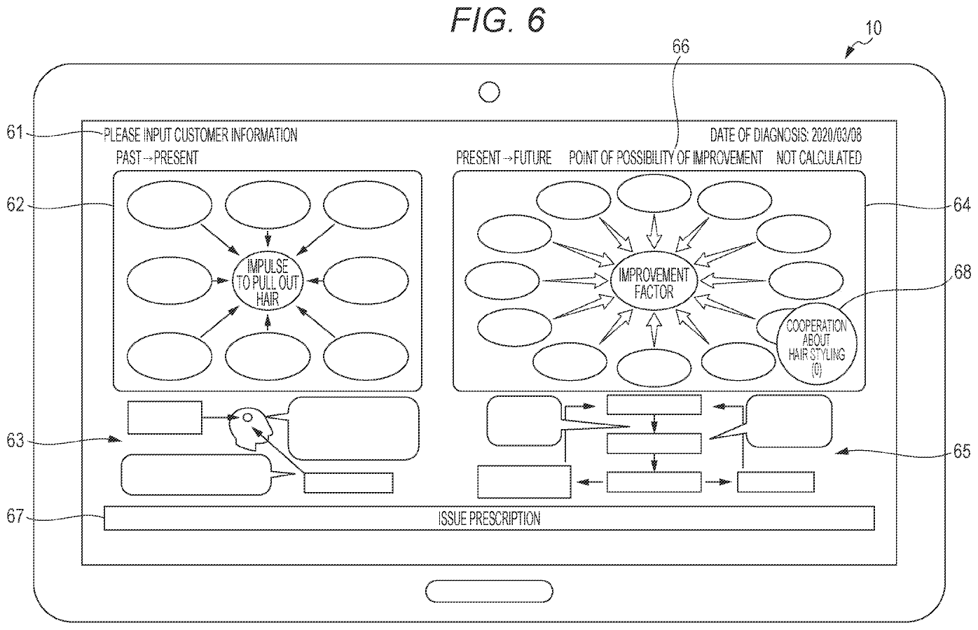

FIG. 6 to FIG. 8 are the customer diagnostic screens displayed on the user terminal 10. The customer diagnostic screen is an exclusive screen for an individual customer having various buttons and display fields. The screens shown in the drawings are only examples, needless to say, various modifications for details are possible.

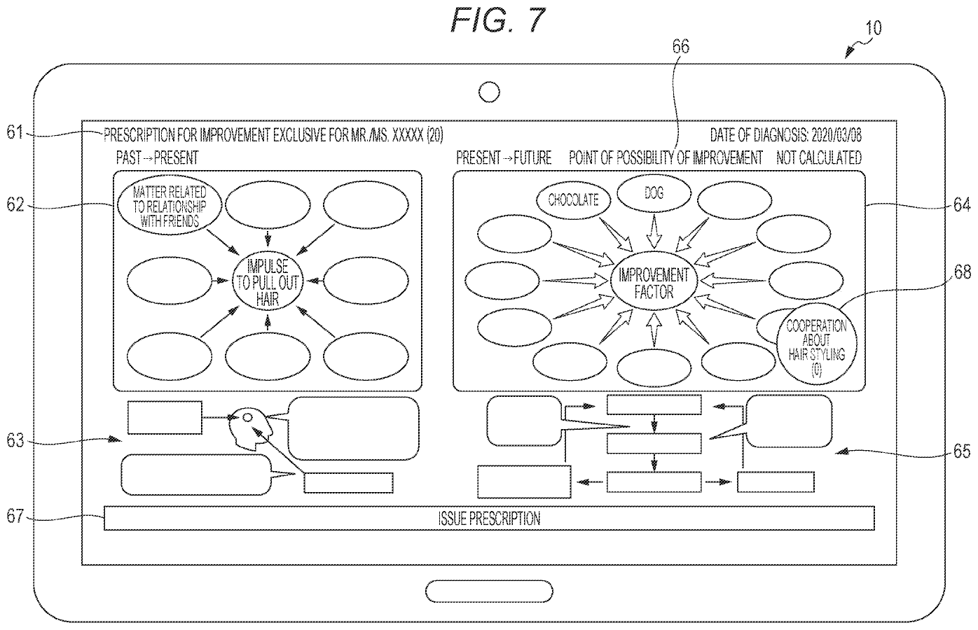

Firstly, when the hairdresser clicks a customer information input unit 61 on the customer diagnostic screen shown in FIG. 6, a screen inputting the customer information is displayed. Thus, by inputting the customer information such as the name, the date of birth, and the gender, the screen reflected with the customer information is displayed (see FIG. 7).

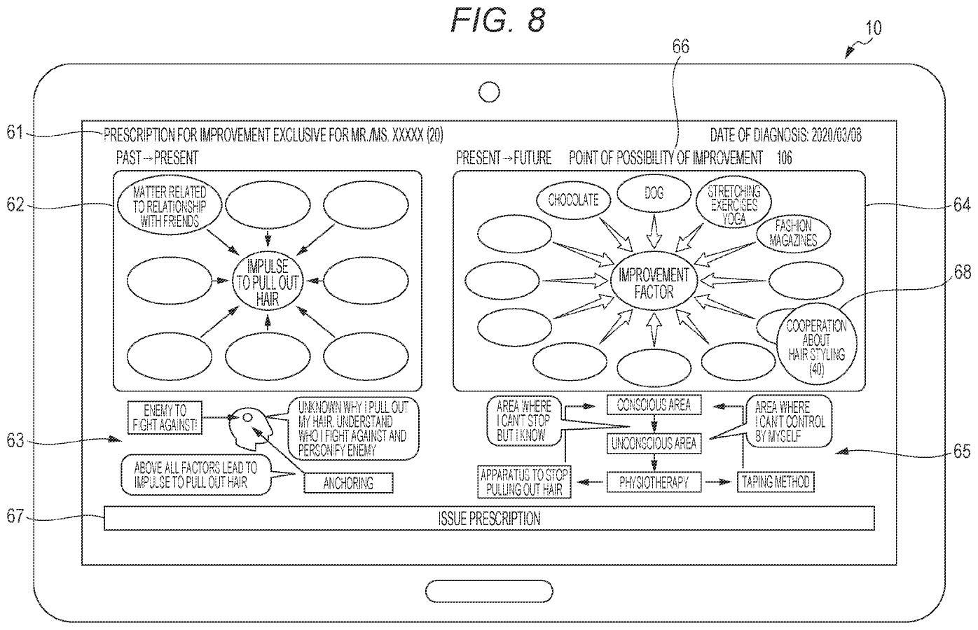

Subsequently, a hearing from the customer is performed. As a result of the hearing, where the matter related to a relationship with friends is a possible cause of the impulse factor of pulling out hair, that matter is inputted in an input unit of the impulse factor of pulling out hair 62 (see FIG. 7). Further, where chocolate and dogs are the improvement factors, these matters are inputted in an input unit of the improvement factor 64 (see FIG. 7). Furthermore, when stretching exercises, yoga, and fashion magazines are the improvement factors, these matters are inputted in an input unit of the improvement factor 64 (see FIG. 8). In this manner, methods of inputting the impulse factors of pulling out hair and the improvement factors are various including clicking predetermined fields on the screen (fields illustrated by circles in the drawings), but not limited thereto.

When the hairdresser clicks on a first display field 63, an explanatory diagram regarding the impulse to pull out hair is displayed in the hearing. When the hairdresser clicks on a second display field 65, an explanatory diagram regarding the improvement factor is displayed (see FIG. 8). These explanatory diagrams may be common contents for all the customers.

The hearing is preferably performed in a following order; (1) to input the impulse factors of pulling out hair, (2) to display the explanatory diagram of the impulse to pull out hair, (3) to input the improvement factors, and (4) to display the explanatory diagram of the improvement factors. In such an order, how to perform the hearing hardly causes differences between the members.

It is possible to change how to perform the hearing from time to time, but the user terminal 10 preferably accepts input of the improvement factor only after the impulse factor of pulling out hair is inputted. In such an order, the customer's sufferings are brought out before an explanation of the improvement factors is given, thereby allowing for enhancing the customer's effort to improve trichotillomania.

Subsequently, by clicking a diagnostic score request unit 66 on the customer diagnostic screen in. FIG. 7, the diagnostic score (106 in this example) is displayed as shown in FIG. 8. Then, by clicking a prescription issuance button 67. a prescription such as in a portable document format (PDF) file is issued and is printable by using the user terminal 10. Contents of the prescription are basically the same as contents of the customer diagnostic screen in FIG. 8.

Further, a diagnostic score adjusting unit 68 where the hairdresser inputs the diagnostic score manually may be provided in the customer diagnostic screen (see FIG. 6 and FIG. 7). As shown in FIG. 8, the diagnostic score adjusting unit 68 makes an experience value of the hairdresser (40 points in this example) possible to reflect on the diagnostic score.

[Statical Data]

The inventor has attempted to improve trichotillomania for more than 2,000 customers in total with a unique method in barbershops and hair salons and collected statistics. Detailed explanation of the unique method is omitted here, but wigs, and hair growth and/or hair restoration may be included.

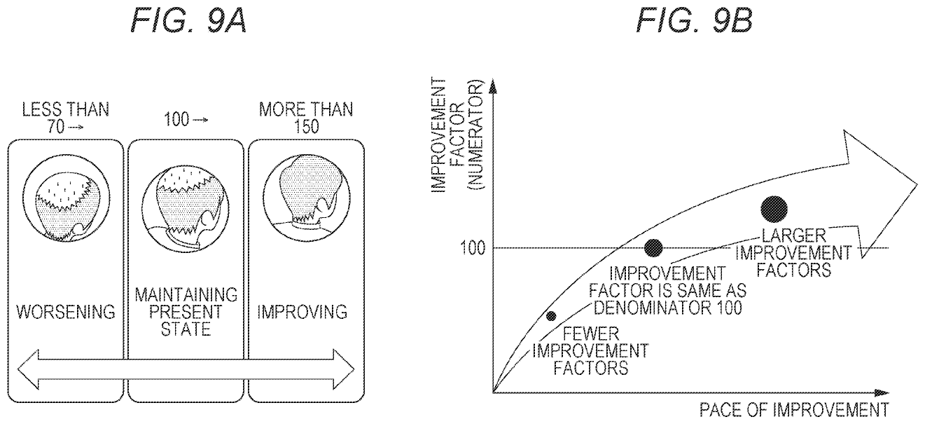

FIG. 9A and FIG. 9B are explanatory diagrams of the statical data according to the embodiment. To simplify descriptions herein, contents of the statistical data are conceptually described.

FIG. 9A is a schematic diagram showing a relation between the diagnostic scores and the possibility of improvement. As shown in FIG. 9A, it is made clear that a customer having the diagnostic score of 70 or less than 70 points tends to worsen trichotillomania. It is also made clear that a customer having the diagnostic score of around 100 points tends to maintain a present state of trichotillomania. Further, it is made clear that a customer having the diagnostic score of 150 or more than 150 points tends to drastically improve trichotillomania. Specifically, an improvement ratio of the customers who have the diagnostic score of 150 or more than 150 points exceeds 90% in one year (nine customers out of ten are improved). FIG. 9B is a graph showing a relation between the improvement factor and a pace of improvement. As shown in FIG. 9B, larger the number of the improvement factors are, faster the pace of improvement is. Suppose that a denominator (the impulse to pull out hair) is 100 points, it is preferable to aim for 150 points for a numerator (the improvement factor). For example, “10 points by snack+10 points+10 points+10 points+20 points by music+20 points by game+30 points by pet animals+20 points by comics+20 points by interesting videos” are preferable. In this example, 150 points of the improvement factors are larger than 100 points of the impulse to pull out hair so that the pace of improvement is faster.

As described above, this system enables the possibility of improving trichotillomania to be quantified and visualized. Therefore, it makes possible to clearly suggest that a person is better to consult a hospital or a barbershop or a hair salon relating to improvement of trichotillomania. In this example, when the diagnostic score is less than 150, a person preferably consults the hospital, and when the diagnostic score is 150 or more than 150, the person preferably consults the barbershop or the hair salon.

It is said that the number of people who suffer from trichotillomania in Japan is more than three million. Under the circumstances, spreading the system to the barbershops and the hair salons becomes highly valuable. Herein, it is exemplified that the system is applied in the barbershop or the hair salon, but the system may be applicable in medical institutions such as a hospital. When the system is applied in the medical institutions, an effect of reducing or eliminating medical treatments can be expected.

[Configuration of Hardware]

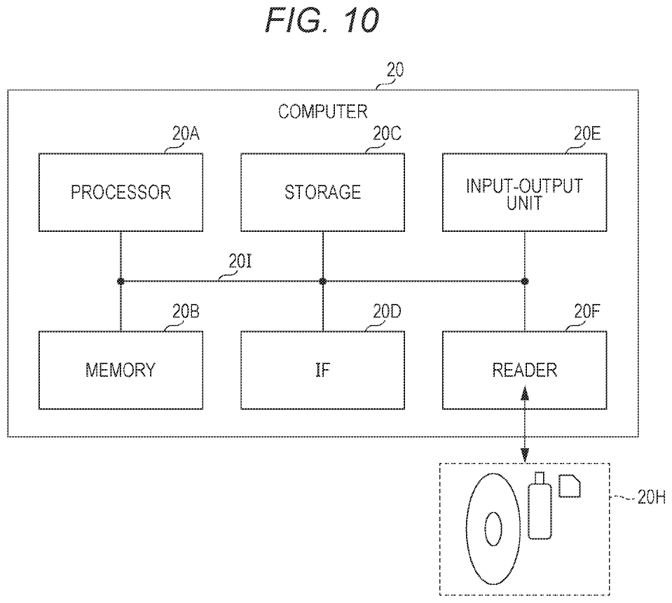

FIG. 10 shows a configuration of hardware of the diagnostic device for improving trichotillomania 20. As shown in FIG. 10, the diagnostic device for improving trichotillomania 20, which is a generic computer, where, for example, a processor 20A, a memory 20B, a storage 20C, an interface (IF) 20D, an input-output unit 20E, and a reader 20F are communicably interconnected via a bus 20I.

The processor 20A is an arithmetic processing unit that performs various controls and computing operations. The processor 20A achieves various functions by executing a program stored in such as the memory 20B, the storage 20C, a storage medium 20H, and a read only memory (ROM) (not shown).

The memory 20B is a storing unit to store various data and programs. For example, a volatile memory such as a random access memory (RAM) is applicable to the memory 20B.

The memory 20C is a storing unit to store various data, programs, and so forth. For example, a magnetic disc unit such as a hard disk drive (HDD), a semiconductor drive unit such as a solid state drive (SSD), and a non-volatile memory such as a flash memory are applicable to the storage 20C.

The interface (IF) 20D controls communications between other terminals. For example, an adapter conforming to a local area network (LAN), a fiber channel (FC), an InfiniBand, and so forth are applicable to the IF 20D.

The input-output unit 20E is an input unit to input data and an output unit to output data. For example, a mouse, a keyboard, and so forth are applicable to the input unit. A display, a printer, and so forth are applicable to the output unit.

The reader 20F is a unit that reads data and a program stored in the computer-readable storage medium 20H. For example, a non-temporal storage medium such as a magnetic/optical disc and a flash memory are applicable to the reader 20F.

As described above, a generic computer can be used for the diagnostic device for improving trichotillomania 20. The same applies to hardware configurations of the user terminal 10 and the administrative terminal 30.

The diagnostic device for improving trichotillomania 20 that is configured on a piece of hardware is exemplified in FIG. 12, but it is not limited thereto. For example, the storage 20C (corresponds to the storage unit 25 in FIG. 2) may be configured on another hardware,

[Characteristic Configuration]

As described above, the diagnostic device for improving trichotillomania 20 according to the embodiment is the device to diagnose the possibility of improvement of trichotillomania that includes the customer information storage unit 25A, the diagnostic information storage unit 25B, and the master improvement factor storage unit 25C, the diagnostic score calculation unit 22, and the diagnostic result issuing unit 23. The customer information storage unit 25A stores the customer information when the customer information including the customer ID is inputted from the user terminal 10 that is communicably connected via the network N. The diagnostic information storage unit 25B stores the impulse factor of pulling out hair as the diagnostic information when the impulse factor of pulling out hair including the customer ID is inputted, and the factor for improvement is stored as the diagnostic information when the factor for improvement including the customer ID is inputted. The master improvement factor storage unit 25C preliminarily makes the improvement factor and the points correspond to each other and stores such data. When the user terminal 10 requests the diagnostic score by specifying the customer ID, the diagnostic score calculation unit 22 extracts all of the improvement factors based on the customer ID, extracts the points corresponding to all of the improvement factors from the master improvement factor storage unit 25C, and calculates the diagnostic score, which is the quantified possibility of improvement of trichotillomania, by adding all of the extracted points. The diagnostic result issuing unit 23 issues the prescription with the diagnostic score when the user terminal 10 requests the issuance of the prescription (the diagnostic result) by specifying the customer ID. In this manner, the possibility of improving trichotillomania can be quantified and visualized.

Additionally, the point updating unit 24 that updates the points stored in the master improvement factor storage unit 25C by using a different algorithm for every category of the improvement factor may be provided. That allows the points to be optimized by every category of the improvement factor as needed, thereby enabling the diagnosis of the possibility of improving trichotillomania at high accuracy.

Alternatively, responding to the boundary points of the diagnostic score where trichotillomania is improved in high probability in the statical data obtained in the barbershops and/or hair salons, the point updating unit 24 may allocate the points to be stored in the master improvement factor storage unit 25C. When the boundary points of the diagnostic score are changed, the allocation of the points to the master improvement factor storage unit 25C can be interlocked in response to a change.

Additionally, the user terminal 10 may be controlled to accept the input of the improvement factor only after the impulse factor of pulling out hair is inputted. In such an order, sufferings of the customers are brought out before the explanation of the improvement factor is given, thereby allowing for enhancing the customer's effort to improve trichotillomania.

According to the present invention, a diagnostic method of improving trichotillomania can be achieved by including respective characteristic function units provided in the diagnostic device for improving trichotillomania 20 as respective steps, or a diagnostic program for improving trichotillomania where these steps are executed in a computer can be achieved.

It is also possible to configure the system by the user terminal 10 alone, when the user terminal 10 includes the characteristic functions provided in the diagnostic device for improving trichotillomania 20. It is needless to say that the program installed on the user terminal 10 is provided via the network N.

[Other Embodiments]

Though the embodiments of the present invention are described herein, parts of the descriptions and drawings disclosed herein are examples, but not limited thereto. The disclosure is to allow persons skilled in the art to know various alternative embodiments and operational technologies.

What is claimed is:

1. A diagnostic device for improving trichotillomania, comprising:
a storage unit for information of a person to be diagnosed configured to store the information of the person to be diagnosed when the information of the person to be diagnosed including an ID of the person to be diagnosed is inputted from a user terminal that is communicably connected via a network;
a diagnostic information storage unit configured to store an pulse factor of pulling out hair as diagnostic information when the impulse factor of pulling out hair including the ID of the person to be diagnosed is inputted, and an improvement factor is stored as the diagnostic information when the improvement factor including the ID of the person to be diagnosed is inputted;
a master improvement factor storage unit configured to preliminarily make the improvement factor correspond to a point and store the improvement factor with the point;
a diagnostic score calculation unit configured to calculate a diagnostic score that is a quantified possibility of improving trichotillomania by extracting all of the improvement factors based on the ID of the person to be diagnosed from the diagnostic information storage unit, by extracting the points corresponding to all of the improvement factors from the master improvement factor storage unit, and by adding all of the points that are extracted, when the user terminal requests the diagnostic score with the ID of the person to be diagnosed specified;
a diagnostic result issuing unit configured to issue a diagnostic result including the diagnostic score when the user terminal requests an issuance of the diagnostic result with the ID of the person to be diagnosed specified; and
a point updating unit configured to update the point stored in the master improvement factor storage unit by using a different algorithm for every category of the improvement factor.

2. The diagnostic device for improving trichotillomania according to claim 1, wherein the point updating unit is configured to allocate the point to be stored in the master improvement factor storage unit in response to boundary points of the diagnostic score where trichotillomania is to be improved in a high probability in accordance with statical data diagnosed in a barbershop and a hair salon.

3. A diagnostic method of improving trichotillomania by using a computer, comprising the steps of:
storing information of a person to be diagnosed including an ID of the person to be diagnosed in a storage unit for the information the person to be diagnosed when the information of the person to be diagnosed including the ID of the person to be diagnosed is inputted;
storing an impulse factor of pulling out hair as diagnostic information in a diagnostic information storage unit when the impulse factor of pulling out hair including the ID of the person to be diagnosed is inputted, and storing an improvement factor as the diagnostic information in a diagnostic information storage unit when the improvement factor including the ID of the person to be diagnosed is inputted;
storing the improvement factor with a point in a master improvement factor storage unit by preliminarily making the improvement factor correspond to the point;
calculating a diagnostic score that is a quantified possibility of improving trichotillomania by extracting all of the improvement factors based on the ID of the person to be diagnosed from the diagnostic information storage unit, by extracting the points corresponding to all of the improvement factors from the master improvement factor storage unit, and by adding all of the points that are extracted, when the user terminal requests the diagnostic score with the ID of the person to be diagnosed specified;
issuing a diagnostic result including the diagnostic score when the user terminal requests an issuance of the diagnostic result with the ID of the person to be diagnosed specified; and
updating the point stored in the master improvement factor storage unit by using a different algorithm for every category of the improvement factor.
